(19) United States
(12) Patent Application Publication
(10) Pub. No.: US 2011/0070204 A1
(43) Pub. Date: Mar. 24, 2011

(54) MEDIUM FOR HANDLING AND STORING BIOLOGICAL TISSUES OF THE MUSCULOSKELETAL SYSTEM OUTSIDE AN ORGANISM

(76) Inventor: Ilan Elias, Philadelphia, PA (US)

(21) Appl. No.: 12/884,394

(22) Filed: Sep. 17, 2010

Related U.S. Application Data
(60) Provisional application No. 61/244,512, filed on Sep. 22, 2009.

(51) Int. Cl. A61K 35/34; C12N 5/02; C12N 5/077

(52) U.S. Cl. 424/93.7; 435/404; 435/405; 435/374; 435/325; 435/366

(57) ABSTRACT

The present disclosure relates to a medium for handling, including storing, cultivating, coating, impregnating and/or preserving musculoskeletal tissues such as bone, cartilage, tendon, muscle, nerve, ligament, blood vessel, skin, fascia, bursa and joint capsule tissue or cells, wherein the medium is an aqueous solution comprising hyaluronan and a first saccharide, polyol, or combination thereof. Further disclosed are methods of handling musculoskeletal tissue or cells or preserving the viability of these tissues or cells using this medium as well as the preserved tissues or cells.

### CROSS-REFERENCE TO RELATED APPLICATIONS

This application is a nonprovisional application of U.S. Provisional Application No. 61/244,512 filed on Sep. 22, 2009, which is incorporated herein by reference in its entirety.

### FIELD OF THE DISCLOSURE

Disclosed herein is a medium for handling, including storing, cultivating, coating, impregnating and/or preserving musculoskeletal tissues such as bone, cartilage, tendon, muscle, nerve, ligament, blood vessel, skin, fascia, bursa and joint capsule tissue or cells, as well as methods of using the medium.

### BACKGROUND

It is estimated that currently over 600,000 bone graft procedures are performed each year in the United States of America, and approximately 2.2 million worldwide. In plastic, maxillofacial, and orthopaedic surgery procedures, the use of autologous bone, also referred as osseous autograft, is common when bone grafts are required to augment bone defects or to perform joint fusions. Even when an allograft is used in surgery, it is usually augmented with autograft. Alternatives to autografts are allograft bone, demineralized bone matrix, recombinant growth factors (e.g. bone morphogenic proteins) and synthetic phospho-calcic materials such as hydroxyapatite and β-tricalcium phosphate.

In the past, the prevailing approach in biomaterials research has been to develop artificial implant materials having optimized mechanical properties and good tissue compatibility. However, no artificial replacement material can match the quality of the body itself For this reason, the repair of defects using autologous cells or tissues represents a further important option for reconstruction of defects. Bodily cells or tissues are removed from a part of the body under less stress, and are reused in the region of the defect in order to trigger new tissue formation at that site. This approach has found increasing acceptance in recent years, primarily due to the rapid development of research in the fields of regenerative medicine and especially musculoskeletal (MSK) tissue engineering. It is thus possible for missing or damaged tissue to be regenerated instead of merely repaired.

One reason for choosing autologous tissue transplants is that they do not cause an immune reaction in the patient. Also, the risk of external infection resulting from viral or bacterial contamination of the donor tissue is minimized. Perhaps more importantly, freshly harvested autologous bone, due to its living cells, is the only kind of graft that provides all three of the fundamental physiological properties of bone: osteogenicity, osteoinductivity, and osteoconductivity. Furthermore, autograft tissue is amenable to revascularization. However, the autograft provides these physiological properties only if it is freshly harvested and immediately implanted. Otherwise, the autograft has to be optimally maintained over the entire period from harvesting to re-implantation, with no damage to the living cells, so that it is possible for the transplant to heal completely and ensure the long-term success of the transplant. The presence of vital osteogenic cells is frequently advocated as the basic requirement for the success of autografts.

During a surgical procedure, it is frequently not possible to immediately re-implant the harvested bone. Usually, there are two different surgical sites: one site where bone is harvested from the patient (e.g., the iliac crest) and a different second site where the autograft is implanted (e.g. spine). In some cases, especially when complications occur during surgery, there can be a prolonged time interval between harvesting and re-implantation. During this interval, common practice among surgeons and assisting personnel is to keep this harvested bone in the operating room at room temperature, either in a jar or dish, with or without saline, or covered in a moist surgical cloth or towel. During this delay and outside the organism, which means in an un-physiologic site, necrosis and apoptosis of bone and bone marrow cells can occur rapidly in the harvested autograft, as the cells are deprived of blood, oxygen and essential metabolites such as saccharides. Thus, when re-implanted, decreased osteogenicity and osteoinductivity of the autograft result and thus may cause improper graft healing or even graft failure, which will require revision surgery.

Because there are currently no standardized methods for temporarily storing autologous tissue transplants, and research findings are still lacking for the most part, clinical practice relies principally on information obtained from the individual experience. Storage of transplants at room temperature under moist, sterile surgical towels, which aims to primarily prevent the cells from drying out, has become the established procedure. Another option is to store the transplants in sterile isotonic saline solution (0.9% NaCl), optionally with the addition of an antibiotic. More preferable than the saline solution is a Ringer's solution or Ringer's lactate solution with an antibiotic, which in comparison to the saline solution contains additional calcium and potassium and therefore essentially matches the composition of blood plasma. The established short-term storage of tissue transplants either under moist, sterile towels or in a saline solution (isotonic saline solution, or Ringer's solution or Ringer's lactate solution) at room temperature fails to actively support the cells and is thus prone to loss of a significant number of cells due to cellular necrosis and apoptosis.

As autologous material is limited in amount and prone to cell death in an un-physiological site (e.g. during graft patency), it is necessary to improve short-term storage of these tissues outside an organism in order to improve tissue quality and to avoid complications in the patient. Accordingly, what is needed is to provide an optimized medium for handling of autologous graft tissue to provide for a better cell survival rate and thus higher-quality transplants, and to avoid cell death such as necrosis and apoptosis in the autograft while being outside an organism (ex vivo).

### SUMMARY

Thus, in a first aspect, a medium for handling tissues or cells of the musculoskeletal system comprises an aqueous solution of hyaluronan and a first saccharide, polyol, or combination thereof, wherein the first saccharide is not hyaluronan. In one embodiment, the medium is for handling the musculoskeletal tissue or cells outside an organism, that is, ex vivo or extracorporeal.

In a second aspect, a method for storing, maintaining, cultivating, coating, impregnating and/or preserving musculoskeletal tissue ex vivo comprises contacting the tissue with the medium disclosed herein. In one embodiment, this method further comprises obtaining the tissue or cells prior to contacting.

In a further aspect, a method for maintaining the viability of musculoskeletal tissue or cells ex vivo comprises contacting the tissue or cells with a medium disclosed herein. This method may further include maintaining the tissue in contact with the medium for a period of time.

In a still further aspect, also included is the use of the medium disclosed herein for handling, including storing, maintaining, cultivating, coating, impregnating and/or preserving musculoskeletal tissue or cells ex vivo.

In still another aspect, an ex vivo preserved biological tissue comprises musculoskeletal tissue and/or cells preserved within a medium as disclosed herein.

### DETAILED DESCRIPTION

It has been unexpectedly found by the inventor herein that an aqueous medium that includes hyaluronan (HA) and a first saccharide, polyol, or combination thereof, wherein the first saccharide is not hyaluronan, can increase the survival rate of musculoskeletal tissue, for example of bone, cartilage, tendon, ligament, muscle, nerve, blood vessel, skin, fascia, bursa and/or joint capsule tissue or cells when residing extracorporeal, i.e., outside the organism, storage, maintenance, cultivation, coating, impregnation and/or preservation, by minimizing the number of cells undergoing cell death (necrosis and/or apoptosis) in comparison to other storage media for example in comparison to storage under moist surgical towel. This medium thus provides for higher cell survival rate with higher tissue quality and avoids complications connected to a loss of viability of the isolated cells when re-implanted into an organism. As used herein, ex vivo or extracorporeal means in an artificial or un-physiologic environment outside of a living organism, or the like.

In a first aspect, a medium for handling musculoskeletal tissues comprises an aqueous solution of hyaluronan and a first saccharide, polyol, or combination thereof, wherein the first saccharide is not hyaluronan.

In one embodiment, the musculoskeletal tissue is from bone (including periost, cortical and trabecular bone with bone marrow), cartilage (including hyaline- and fibro-cartilage), tendon, ligament, muscle, connective tissue, nerve, blood vessel (including arteries and veins), skin, fascia, bursa sac, joint capsule, or a combination thereof.

“Saccharides” are carbohydrates, including monosaccharides, disaccharides, oligosaccharides, polysaccharides, glycosaminoglycans, or a combination thereof.

“Monosaccharides” are monomeric carbohydrate molecules, i.e., a polyalcohol comprising an aldehyde (aldose) or keto (ketose) group. Exemplary monosaccharides are hexoses, such as glucose, allose, altrose, gulose, idose, talose, mannose, galactose, fructose, psicose, sorbose, and tagatose, or pentoses, such as ribose, arabinose, xylose, lyxose, ribulose, and xylulose.

“Disaccharides” are molecules consisting of two monosaccharides that are linked by a glycosidic bond. Disaccharides include, but are not limited to sucrose, lactose, maltose, trehalose and cellobiose.

“Oligosaccharides” are polymers of 3-20, for example 3-10 monosaccharide units that are linked by glycosidic bonds. Exemplary oligosaccharides include, but are not limited to raffinose, maltotriose, melzitose, stachyose, and nystose.

“Polysaccharides” are polymeric carbohydrate structures, formed of repeating units of mono- or disaccharides linked by glycosidic bonds. Typically, polysaccharides comprise 21 to about 3,000 monosaccharide units. Exemplary polysaccharides include, but are not limited to glucans, such as alpha- or beta-glucans; amylase; amylopectin; glycogen; dextran; cellulose; chitin; chitosan and chitosan derivatives (e.g., carboxymethyl-chitosan, trimethyl-chitosan); agar; starch; or a combination thereof As used herein, the term citosan includes chitosan and its derivatives.

A “polyol” is a polyalcohol, i.e. a molecule having two or more hydroxy groups. Exemplary polyols are those derived from monosaccharides (sugar alcohols) by replacement of the aldehyde or keto group with a hydroxy group, such as glycerol, maltitol, mannitol, or sorbitol, or glycol, and combinations thereof.

“Hyaluronan”, “HA”, and “hyaluronic acid”, as used interchangeably herein, refer to a polymer of disaccharides, themselves composed of D-glucuronic acid and D-N-acetylglucosamine, linked together via alternating β-1,4 and β-1,3 glycosidic bonds. In one embodiment, hyaluronan is 25,000 disaccharide repeats in length. Polymers of hyaluronan can range in size from 5,000 to 20,000,000 Da in vivo. The average molecular weight of hyaluronan in human synovial fluid is 3-4 million Da. The term also includes all salts of hyaluronic acid, such as sodium, potassium, zinc or iron salts.

“Bone tissue” is osseous tissue, i.e. a mineralized connective tissue. The bone tissue is formed by bone-forming cells called osteoblasts, which produce and deposit osteoid, a matrix of Type I collagen. Osteoblasts also release calcium, magnesium, and phosphate ions, which chemically combine and harden within the matrix into the mineral hydroxyapatite. Bone tissue may contain blood vessels and nerves that service the bone. Bone tissue may comprise osteoblasts, and bone-resorbing cells, the osteoclasts. Furthermore, bone tissue may comprise bone marrow cells as well as periosteum.

“Cartilage tissue” is a specific type of dense connective tissue composed of specialized cells called chondrocytes that produce an extracellular matrix composed of collagen, proteoglycans and elastin fibres. Unlike other connective tissues, cartilage does not contain blood vessels or nerves. As used herein, cartilage tissue includes hyaline cartilage (e.g. articular cartilage) and fibrocartilage (e.g. meniscus, intervertebral disc).

“Muscle tissue” is contractile tissue usually derived from the mesodermal layer of embryonic germ cells. As used herein, muscle tissue includes skeletal and smooth muscles.

“Nerve tissue” or “nerves” are enclosed, cable-like bundles of peripheral axons coated with myelin and may comprise neurons and/or Schwann cells.

“Ligament tissue” is tissue of articular ligaments, i.e. fibrous tissue that connects bones with each other to form a joint. A ligament is a short band of tough fibrous dense regular connective tissue composed mainly of long, stringy collagen fibers.

“Tendon tissue” is tissue of a tendon, i.e., a band of fibrous connective tissue that connects muscle to bone. Tendons are mostly composed of parallel arrays of collagen fibers, mostly type I collagen, closely packed together and further comprise minor amounts of elastin, proteoglycans and inorganic ions, such as calcium.

“Joint capsule tissue” is a tissue of a joint capsule (articilar capsule) which is an envelope surrounding a synovial joint and defining an intraarticular joint space. The joint capsule tissue is comprised of an outer fibrous layer and an inner secreting layer, which is the synovial membrane.

“Connective tissue” is a fibrous tissue that is found in the musculoskeletal system and connects other tissues with each other.

“Blood vessel tissue” is a tissue of blood vessel comprised of elastic hollow tubes that carry blood from the heart to the musculoskeletal system (arteries) and from the musculoskeletal system to the heart (veins). The walls of the blood vessels consists of three layers the tunica intima, the tunica media, and the tunica adventitia.

“Fascia tissue” is a strong connective tissue that surrounds muscles, groups of muscles, blood vessels, and nerves, binding those structures together. Fascia tissue is found throughout the musculoskeletal system.

“Skin tissue” is a tissue of skin, which composes the epidermis, the dermis, connective tissue and the subcutaneous fat layer. Skin tissue is the outer-most protective layer of the musculoskeletal system.

In one embodiment, the first saccharide, polyol, or combination thereof comprises a monosaccharide, such as fructose, for example D-fructose. The concentration of the monosaccharide is about 500 to about 10,000 mg/L, about 1,000 to about 7,000 mg/L, about 2,000 to about 5,000 mg/L or about 3,000 to about 4,000 mg/L. In a specific embodiment, the monosaccharide is D-fructose and the concentration of the monosaccharide is about 3,600 mg/L.

In certain embodiments, the medium optionally comprises a second saccharide or polyol selected from the group consisting of glucose, mannitol and sorbitol, wherein the second saccharide is not hyaluronan. In one embodiment, the resulting medium would thus comprise hyaluronan, fructose and a second monosaccharide selected from the group consisting of glucose, mannitol and sorbitol. In some embodiments, the glucose is D-glucose. The second saccharide, polyol, or combination thereof, when present, is present in the same concentration as the first saccharide or polyol.

The hyaluronan is present in the medium at a concentration of about 500 mg/L to about 20,000 mg/L, about 1,000 to about 10,000 mg/L, or about 1,000 to about 2,500 mg/L. In one embodiment the hyaluronan is present at 0.05% (wt/vol) to 2% (wt/vol), preferably 0.1% (wt/vol) to 1% (wt/vol). In some embodiments the hyaluronan is present in the medium at a concentration of 0.1% (wt/vol) and of 0.25% (wt/vol) as shown in Tables 1, 2 and 3.

In some embodiments the hyaluronan has a mean molecular weight of about 100,000 Da to about 5,000,000 Da, about 250,000 to about 3,000,000 Da, about 500,000 to about 2,000,000 Da, or about 1,600,000 Da.

In specific embodiments, the medium comprises about 500 mg/L to about 20,000 mg/L hyaluronan and about 500 to about 10,000 mg/L fructose. The fructose is preferably D-fructose.

In one embodiment, the medium comprises about 1,000 mg/L to about 2,500 mg/L hyaluronan and about 2,000 to about 4,000 mg/L D-fructose.

In another embodiment, the medium comprises about 1,000 mg/L or 2,500 mg/L hyaluronan and about 3,600 mg/L D-fructose.

In the media comprising hyaluronan and fructose, the hyaluronan preferably has a mean molecular weight of about 100,000 Da to about 4,000,000 Da, more preferably about 1,600,000 Da.

In one embodiment, the medium comprises about 0.05 wt % to about 2.0 wt % chitosan.

In one embodiment, the medium comprises about 0.05 wt % to about 2.0 wt % beta-glucan.

In another embodiment, the medium comprises at 0.05% (wt/vol) to 2% (wt/vol) hyaluronan, 500 to 10,000 mg/L fructose, and 0.01 wt % to 2.0 wt % chitosan.

In another embodiment, the medium comprises at 0.05% (wt/vol) to 2% (wt/vol) hyaluronan, 0.05wt % to 2.0 wt % beta-glucan, and 0.01wt % to 2.0 wt % chitosan.

In some embodiments, the medium further comprises one or more compounds selected from the group consisting of buffer substances, salts, vitamins, amino acids, lipids including phospholipids, antibiotics, anti-fungal drugs, corticosteroids, growth factors including platelet-derived growth factor (PDGF), detergents, pH indicators, and combinations thereof.

Exemplary buffer substances include disodium hydrogenphosphate (Na₂HPO₄), dipotassium hydrogenphosphate (K₂HPO₄), sodium dihydrogenphosphate (NaH₂PO₄), potassium dihydrogenphosphate (KH₂PO₄) sodium bicarbonate (NaHCO₃), and combinations thereof.

Exemplary salts include sodium chloride, potassium chloride, sodium citrate, magnesium chloride, calcium chloride, ferrous nitrate, magnesium sulfate, and combinations thereof.

Exemplary amino acids include L-arginine, L-cysteine, L-glutamine, glycine, L-histidine, L-isoleucine, L-leucine, L-lysine, L-methionine, L-phenylalanine, L-serine, L-threonine, L-tryptophan, L-tyrosine, L-alanine, L-asparagine, L-glutamic acid, L-aspartic acid, L-proline, L-valine, and combinations thereof.

Exemplary vitamins include choline, pantothenate, D-panthenol, myo-inositol, folic acid, nicotinamide, pyridoxal, riboflavin, thiamine, ascorbic acid, retinol, ergocalciferol, cholecalciferol, tocopherol, ubiquinone, phylloquinone, menaquinone, and combinations thereof.

Exemplary phospholipids include phosphatidylcholine, phosphatidylserine, phosphatidylethanolamine, and combinations thereof.

In some embodiments of the invention, the medium further comprises allantoin.

According to one embodiment, the pH of the medium is about 6 to about 9, specifically about 7 to about 8, for example pH 7.4.

In one embodiment, the medium is sterile and can therefore be used during surgical procedures in the operating room. The medium can either be compounded in a sterile manner with sterile components or it can be compounded and then later autoclaved to make it sterile. Furthermore, the medium can be sterilized using other sterilization methods for example exposure to radiation (e.g. gamma rays), ethylene oxide, or the sterile filtration method.

The handling of the tissue or cells using the medium described herein includes storing, maintaining, cultivating, coating, impregnating and/or preserving.

In another aspect, included herein is a method for handling, including storing, maintaining, cultivating, coating, impregnating and/or preserving, musculoskeletal tissue, comprising contacting the tissue with the medium of the invention described above.

In one embodiment, the musculoskeletal tissue consists of bone (including periost, cortical and trabecular bone with bone marrow), cartilage (including hyaline- and fibro-cartilage), tendon, ligament, muscle, connective tissue, nerve, blood vessel (including arteries and veins), skin, fascia, bursa sac and joint capsule tissue or cells.

In one embodiment, the method includes obtaining the musculoskeletal tissue. In certain embodiments of the invention, the tissue is obtained by harvesting during surgery.

In one embodiment, the method includes obtaining the musculoskeletal tissue by harvesting of a dead individual.

In one embodiment, the tissue or cells are obtained from a mammalian, preferably human source.

The contacting step may comprise submerging the musculoskeletal tissue in the medium and/or spraying or pipette dripping the medium onto the musculoskeletal tissue.

In one embodiment, the musculoskeletal tissue is ex vivo in a container and is either partially or entirely embedded in the medium.

In some embodiments, the method further comprises transplanting said musculoskeletal tissue into a patient. The patient may be a mammal, for example a human.

In specific embodiments, the patient receiving the musculoskeletal transplant is the same patient from which the transplant has been obtained. In these cases, the transplant is an autograft or auto-transplant.

In other embodiments, the musculoskeletal transplant is harvested from a (either an alive or dead) human being different from the receiving patient (alive). In these cases, the transplant is an allograft or allotransplant.

In other embodiments, the musculoskeletal transplant is harvested from an unlike species (e.g., swine, bovine, monkey). In these cases, the transplant is a xenograft or xenotransplant.

Another aspect also encompasses a method for maintaining the viability of musculoskeletal tissue ex vivo comprising contacting said tissue with a medium as described herein. As used herein, the term “ex vivo” means handling of living cells or tissues taken from an organism and stored, for example in the operating room during surgery or in a laboratory.

In one embodiment, the musculoskeletal tissue is from bone (including periost, cortical and trabecular bone with bone marrow), cartilage (including hyaline- and fibro-cartilage), tendon, ligament, muscle, connective tissue, nerve, blood vessel (including arteries and veins), skin, fascia, bursa sac, joint capsule, or a combination thereof. For example combination of tissues are but not limited to osteochondral graft, femoral head, bone-patellar tendon-bone graft, radial-forarm-free-flap graft, anterolateral-thigh-free-flap graft, fibula-osteocutaneus-free-flap graft, skin-muscle-fat-tissue-vessel-nerve free graft, muscle-tendon graft.

In one embodiment, this method includes maintaining the tissue in contact with said medium for a period of time.

In some embodiments, the temperature in the maintaining step is about 10-34° C., about 11-25° C., or about 17-22° C. or about 4° C. The period of time is about 20 minutes to about 72 hours, for example 30 minutes to about 48 hours or about 1-24 hours, or about 90 minutes.

In a still further aspect, included herein is the use of the medium described herein for handling musculoskeletal tissues ex vivo. The musculoskeletal tissue is from bone, cartilage, tendon, ligament, muscle, nerve, blood vessel, skin, fascia, bursa or joint capsule tissue. The handling includes storing, maintaining, cultivating, coating, impregnating and/or preserving ex vivo tissues or cells.

In still another aspect, included herein an ex vivo preserved biological tissue comprises viable musculoskeletal tissue, such as bone, cartilage, tendon ligament, muscle, nerve, blood vessel, skin, fascia, bursa or joint capsule cells or tissue, within a medium as described herein.

In some embodiments, the tissue is preserved in a hypothermic condition or a physiological condition. The physiological condition comprises a temperature of about 37° C. The hypothermic condition comprises a temperature lower than 35° C., for example about 17-22° C. or about 4° C.

In some embodiments, the preserved biological tissue is a transplant, for example an autologous transplant, an allotransplant or a xenotransplant.

The invention is further illustrated by the following non-limiting examples. As one of ordinary skill in the art will readily appreciate from the disclosure, other compositions of matter, means, uses, methods, or steps, presently existing or later to be developed that perform substantially the same function or achieve substantially the same result as the corresponding exemplary embodiments described herein may likewise be utilized according to the present invention.

### EXAMPLES

### Example of Tissue Sample Preparation

The tissue samples were harvested from a one-year-old calf metacarpals and metatarsals. The fresh calf limbs were delivered immediately after slaughtering from a slaughterhouse and were prepared under sterile laboratory conditions shortly after slaughtering.

First, the skin of the leg was opened lengthwise in the direction of the joint using a general-purpose slicer, the joint was severed, and the hoof and skin were removed. After that, the preparation was thoroughly washed under flowing water and disinfected with ethanol (80%).

At the articular fovea (fovea articularis) of the os compedale (first phalanx) and the condyle (caput metatarsale), the articular cartilage was removed; it had a square shape, with an edge length of about 1 cm and a standard anatomical depth of 1 to 2 mm.

The ends of the tendon fastened to the epiphysis of the os metatarsale are divided into sections of about 1 cm. Due to their anatomy; the tight, parallel-fiber connective tissue pieces have a thickness of 0.5 to 1 cm.

The bone samples were taken from the diaphysis of the os metatarsale. To do this, first the soft tissue was removed from the bone, exposing the substantia compacta. In order not to cause any frictional cell injury or thermal necrosis when the bone samples were taken, this was done using a punch (Perkin-Elmer Deutschland, Überlingen, Germany). The punched-out bone fragments have a cylindrical shape with a diameter of about 2-3 mm and a layer thickness of about 1 mm.

### Storage Series of Tissue Samples

After the tissue samples were prepared and cleaned in PBS solution (4.00 g NaCl-0.9%, 0.10 g KCl, 0.72 g Na₂HPO₄.2H₂O, 0.10 g KH₂PO₄, 500 mL distilled water), they were stored for 90 minutes, 18, 24, or 48 h in five different storage media, and then fixed.

For comparison, a sample of each tissue type was taken and fixed immediately after it was removed.

The tissue samples were stored at room temperature for 90 minutes., 18, 24, or 48 h under moist, sterile surgical towels (moist with NaCl-0.9%), in 0.1 mol/L of sterile-filtered PBS aqueous solution (4.00 g NaCl-0.9% , 0.10 g KCl, 0.72 g Na₂HPO₄.2H₂O, 0.10 g KH₂PO₄, 500 mL distilled water), with and without sterile hyaluronan (0.1%, 0.25%, 0.5%, and 1.0% (TRB Chemedica AG, Haar/Munich, Germany)) and D-fructose (3600 mg/L Sigma-Aldrich Gmbh, Munich, Germany), or MM0 aqueous solution (high GEM without L-glutamine or vitamins (84 mg/L L-arginine HCl, 48 mg/L L-cystein, 30 mg/L glycine, 42 mg/L L-histidine HCl, 104,8 mg/L L-isoleucine, 104,8 mg/L L-leucine, 145,2 mg/L L-lysine HCl, 30 mg/L L-methionine, 66 mg/L L-phenylalanine, 42 mg/L L-serine, 95,2 mg/L L-threonine, 16 mg/L L-tryptophan, 72 mg/L L-tyrosine, 93,6 mg/L L-valine, 264,9 mg/L CaCl₂*2H₂O, 0,1 mg/L Fe(NO₃)₃*9H₂O, 97,7 mg/L MgSO₄, 400 mg/L KCl, 3700 mg/L NaHCO₃, 6400 mg/L NaCl-0.9%, 125 mg/L NaH₂PO₄*H₂O, 3600 mg/L D-fructose, 115 mg/L phenol red sodium salt, 110 mg/L sodium pyruvate) (BIOTECH GmbH, Henningsdorf, Germany), 60 mL FCS (fetal calf serum) (Biochrom, Berlin, Germany), 5 mL penicillin/streptomycin (Biochrom, Berlin, Germany), 5 mL amphotericin B (Biochrom, Berlin, Germany), 5 ml glutamine (Biochrom, Berlin, Germany)). After removal, the cells were fixed.

### Paraffin Embedding and Sectioning of Tissue

After the time periods described above, the tissue samples were removed from their respective test solutions and fixed (10% paraformaldehyde solution (Merck KGaA, Darmstadt, Germany)). The bone samples additionally underwent decalcification for 14 days in EDTA solution (220 g Titriplex® III (Merck KGaA, Darmstadt, Germany), 70 mL of 40% sodium hydroxide solution (pH 7.4), NaOH flakes (Merck KGaA, Darmstadt, Germany), 1 L distilled water. A positive control was prepared in the form of a dental X-ray.

In the following steps of the embedding, all tissue samples were dehydrated by introduction into a graded alcohol series. To do this, the bone, cartilage, and tendon samples were stored at room temperature for 1 h in each of the solutions 50% ethanol, 70% ethanol, and 100% isopropanol. After that, all preparations were stored for two days in cedarwood oil at 40° C. in the incubator until the sample was transparent. Then, the cedarwood oil (Merck KGaA, Darmstadt, Germany) was replaced by paraffin (Paraplast® Plus (Kendall, Mansfield, Mass., USA), and the samples were left in the incubator for another day at 60° C. For each combination of time period and storage medium, two samples were put, together with paraffin, into a prefabricated mold suitable to mount the microtome. Care was taken to embed the preparations in such a way that it was later possible to perform a longitudinal section on the microtome.

After the tissue samples from the incubator had completely cooled, the preparation was cut into serial sections 4 μm thick, which were then put onto glass slides. Finally, the sections were dried on a hotplate.

### Hematoxylin-Eosin Staining of Tissue

In order to be able to evaluate the number and distribution of the cells in the different tissue samples, first all tissue sections were stained with a general histological stain in the form of a hematoxylin-eosin stain. This general stain turns cell nuclei blue and connective tissue pink.

The general staining first required that the tissue sections be deparaffinized. This was done by a graded alcohol series of all samples with xylene (Merck KGaA, Darmstadt, Germany), 100% isopropanol (Merck KGaA, Darmstadt, Germany), 90% ethanol, 80% ethanol, 70% ethanol, and distilled water, over a total time period of 50 minutes. To do this, ten slides were put on a support and each was incubated twice for 10 minutes in 100% xylene, twice for 5 minutes in 100% isopropanol, and for 5 minutes in 90%, 80%, and 70% ethanol.

Then, the cells were stained with Mayer's hematoxylin (1,000 mL distilled water, 1 g hematoxylin (Merck KGaA, Darmstadt, Germany), 0.2 g NaI (Merck KGaA, Darmstadt, Germany), 50 g potassium alum (Merck KGaA, Darmstadt, Germany), 50 g chloral hydrate (Carl Roth Gmbh, Karlsruhe, Germany), 1 g citric acid (Merck KGaA, Darmstadt, Germany)). This involved putting the slides in the staining dish with the hematoxylin solution for 10 minutes, and after that rinsing them under flowing tap water for 15 minutes. Then, plasma staining was done by immersion for 5 minutes in eosin (250 mL of 70% alcohol, 50 mL of eosin stock solution (10 g of eosin in 1,000 mL of 70% alcohol), Eosin (Chroma Gesellschaft Schmid GmbH & Co, Köngen, Germany), 3-4 drops glacial acetic acid. Finally, all samples were rinsed with distilled water and put into a graded alcohol series (5 minutes into each of the solutions 70%, 80%, 90% ethanol, 2×10 minutes 100% isopropanol, 2×10 minutes xylene).

Before the preparations were covered, the stain was randomly checked under the optical microscope. Finally, all preparation sections were completely covered with Eukitt® solution (O. Kindler GmbH & Co. Mikroskopische Gläser, Freiburg im Breisgau, Germany) and coverslips according to size.

Staining Apoptotic and Necrotic Cells of Tissues after Storage

Dead cells within the prepared tissue sections were detected using the TdT-FragEL™ DNA Fragmentation Detection Kit (Calbiochem® (Merck KGaA, Darmstadt, Germany)). This simultaneously allows assessment of two indicators for programmed cell death, apoptosis. First, apoptosis causes histologically recognizable morphological changes in the form of cell shrinkage, pyknosis (condensation/thickening) of the cell nucleus, chromatin condensation, and vesicle formation in the plasma membrane. Moreover, apoptotic endonucleases split the cellular DNA into individual DNA fragments. At their ends, these fragments have free OH-groups, which can be labeled and thus made visible by staining with the TdT-FragEL™ DNA Fragmentation Detection Kit. This allows dead and vital cells to be distinguished far more precisely than histological examination does, since the labeling reaction that is used can identify DNA strand breaks even within morphologically intact cell samples.

The labeling and staining reaction required that the tissue sections first be deparaffinized and rehydrated. This was done by a graded alcohol series of all samples with xylene, 100% isopropanol, 90% ethanol, 80% ethanol, 70% ethanol, and distilled water, over a total time period of 50 minutes. To do this, each slide was incubated twice for 10 minutes in 100% xylene, twice for 5 minutes in 100% isopropanol, and for 5 minutes in 90%, 80%, and 70% ethanol. Finally, the preparations were rinsed with TBS (20 mM Tris pH 7.6, 140 mM NaCl-0.9%). To get small reaction volumes around the preparations, it turned out to be helpful to circle the sample with the Dako Pen (DAKO Diagnostik GmbH, Hamburg, Germany).

To permeabilize the preparations, each section was covered with proteinase K (2 mg/mL proteinase K in 10 mM Tris, pH 8) and Tris (Tris 10 mM, pH 8 (Biomol GmbH, Hamburg, Germany)) in the ratio 1:100, and incubated for 20 minutes. Then, each preparation was rinsed with TBS.

To ensure that the staining was working correctly, a positive control was generated using the supplied control slides and additionally slides of the media preparations. These control samples were incubated for 20 minutes with 1 μg/μL DNase I (DNase I, bovine pancreas (Calbiochem®, Merck KGaA, Darmstadt, Germany) in 1×TBS/1 mM MgSO₄ (Magnesium sulfate 1 mM MgSO₄ in 1×TBS (Merck KGaA, Darmstadt, Germany)) and then rinsed with TBS.

To inactivate the endogenous peroxidases, each slide was covered with 30% hydrogen peroxide and methanol in the ratio 1:3. After incubation for 5 minutes, each slide was rinsed again with TBS.

To carry out the actual labeling reaction, 5×TdT equilibration buffer (1 M sodium cacodylate, 0.15 M Tris, 1.5 mg/mL BSA, 3.75 mM CoCl₂, pH 6.6) was first diluted with distilled water in the ratio 1:5, and added to the sample for 30 minutes.

The following step involved preparing, for each individual slide, a labeling solution from 57.0 μL TdT labeling mixture with 3.0 μL TdT enzyme (25 U TdT (Promega, Madison, Wis.), 1 nmol digoxigenin-11-deoxyuridine-triphosphate (Boehringer Mannheim, Mannheim, Germany) in 400 μl of equilibration buffer (200 mmol/L cacodylat, 25 mmol/L Tris-HCl pH 6.6, 250 μg/ml bovine serum albumin and 2.5 mmol/L cobalt chloride) on ice. After incubation for 30 minutes, the sample was immediately covered with the prepared labeling solution. In addition, each slide was given a Parafilm® (Parafilm M (Pechiney Plastic Packaging) Alcan Singen, Germany) covering, to ensure distribution of the reaction mixture and prevent evaporation losses during incubation. All slides were placed into a moist chamber at 37° C. and incubated for 90 minutes.

To end the labeling reaction, the Parafilm® covering was removed after the incubation time had ended, and the slide was rinsed with TBS. The sample was covered with the stop buffer (0.5 M EDTA, pH 8), incubated for another 5 minutes, and rinsed again with TBS.

To visualize the free OH-groups that were detected by staining, first blocking buffer (4% BSA in PBS) was put onto each sample, and it was incubated for 10 minutes. Next, 50× conjugate (50× peroxidase streptavidin conjugate), diluted with the blocking buffer in the ratio 1:50, was added to all samples without TBS rinsing, and the samples were incubated for another 30 minutes. Then, the DAB solution was prepared from one DAB tablet (3,3′-diaminobenzidine (0.7 mg/tablet)), one H₂O₂ urea tablet (H₂O₂/urea (1.6 mg/tablet)), and 1 mL of tap water. After TBS rinsing, the DAB solution was put onto the preparation and it was incubated for 15 minutes. After that all samples were rinsed with distilled water. Finally, each sample to be stained was covered with the methyl green staining solution (0.3% methyl green) and incubated for another 3 minutes. Last, all preparations were dehydrated twice each in 100% isopropanol and 100% xylene. Before the preparations were covered, the stain was randomly checked under the optical microscope. The last step was to cover each slide with Eukitt® and the appropriate coverslips.

### Optical Microscopy

The pictures of the preparation sections of the HE general stain and the apoptosis stain were taken with the optical microscope Axioplan 2 using the associated camera Axio Cam MRc (Zeiss, Oberkochen, Germany).

For the HE general stain, pictures were taken at 12.5× and 40× magnification to get an overall view of the entire sample. The distribution of the different tissue cells was clear at magnifications of 100× and 200×. At magnifications of 400× and 640×, morphological differences of the cells were visible. For each preparation section of the apoptosis stain, five pictures were taken, each at different places, with a magnification of 200×. In addition, overview pictures of the preparation were taken at magnifications of 12.5× and 40×. For the cartilage sections, it turned out to be useful to take detailed pictures at a magnification of 400×, since the cell morphology had changed.

Counting Living and Apoptotic/Necrotic Cells after Storage of Tissues

For later statistical evaluation of the samples at different fixation times in the respective media, first the number of cells was determined for each individual image that was taken at a magnification of 200×. This involved distinguishing apoptotic and living cells.

The cell count was determined using the software ImageJ (National Institutes of Health, 9000 Rockville Pike, Bethesda, Md. 20892, USA). It allows, with the CellCounter, different marking and counting of several cell types (Type 1: living cells—blue stain marking; type 4: apoptotic cells—magenta stain marking). After the corresponding stain marking is selected in the CellCounter, the program automatically determines the number of cells of each type by counting the mouse clicks with which the cells of this type are marked in the image.

After the counting was completed, each image was separately exported and saved again with the corresponding count markings.

### EXAMPLES

Example of Handling Bone Tissue ex vivo

The proportion of dead bone cells when the tissue undergoes immediate fixation, is 4.5% and is shown for comparison in Table 1.

Storage in moist air: After storage for 90 minutes in moist air, the percentage of dead cells has increased by a factor of almost five as compared with immediate fixation, and after 18 hours it has progressively increased by a factor of more than 10. As expected, the maximum is reached after 48 hours, where 65% of the cells are dead.

Storage in PBS: Tissue stored for 90 minutes in PBS shows an increase in the number of dead cells to 16%, almost a 4-fold increase over immediate fixation. Over the entire period of the experiment, a steady rise can be observed. After 18 hours the number of dead tissue cells is 25%, after 24 hours it is 30%, and after 48 hours it reaches a maximum of 46%.

Storage in nutrient solution (MM0): This nutrient solution shows the second worst result after storage in moist air. After storage for 90 minutes, the number of dead tissue cells increases by a factor of 5 over that which occurs when the tissue undergoes immediate fixation, and after 18 hours it increases by a factor of 7. After 24 hours, the percentage of dead cells was 26%. After 48 hours, a maximum of 52% is reached.

Storage in PBS+0.1%-HA and D-fructose: After storage for 1.5 hours in 0.1% HA, 3 times as many dead cells are present as when the tissue undergoes immediate fixation. After storage for 18 and 24 hours in 0.1% hyaluronan, the percentage of apoptotic cells is approximately 10%. After 48 hours the percentage of dead cells was found to be 22%. Thus, at the end of the series of experiments, the tissues stored in 0.1% hyaluronan contain the fewest apoptotic bone cells.

Storage in PBS+0.25%-HA and D-fructose: The number of tissue cells that are damaged after 90 minutes in 0.25% hyaluronan has increased to 16.3%, thus by a factor of 3.5 in comparison with the initial value. After storage for 18 hours, 7.0% of the cells in the sample were apoptotic. After 24 hours the number of apoptotic cells is 14.4%; after 48 hours, 30.8% of the cells in the sample were dead.

Table 1 shows the mean apoptosis rates for bone tissue in different media in tabular form. Standard deviations are given in brackets.

TABLE 1
Cell death rates for bone tissue stored in different media over time

| | | Storage Media | | | | |
|---|---|---|---|---|---|---|
| | No Storage | | | | PBS + 0.1% | PBS + 0.25% |
| TIME | medium | Moist Air | PBS | MM0 | HA | HA |
| Immediate | 4.5 (1.8) | — | — | — | — | — |
| 1.5 h | — | 21.8 (9.8) | 15.9 (5.4) | 25.1 (12.8) | 15.2 (5.8) | 16.3 (2.3) |
| 18 h | — | 50.6 (21.2) | 25.0 (7.7) | 34.2 (9.8) | 9.6 (5.7) | 7.0 (3.7) |
| 24 h | — | 46.2 (10.4) | 30.1 (11.9) | 26.3 (6.1) | 9.5 (3.6) | 14.4 (13.9) |
| 48 h | — | 65.2 (13.7) | 46.5 (12.0) | 52.2 (21.0) | 22.4 (3.2) | 30.8 (8.8) |

This table shows the mean cell death (apoptosis/necrosis) rates for bone tissue stored in different media over time in % (sd). For example: bone tissue stored in PBS+0.1% HA solution after 24 hours was found to have 9.5% (sd 3.6) dead cells.

Example of Handling of Cartilage Tissue ex vivo

The proportion of dead cartilage cells when the tissue undergoes immediate fixation is 3.1% and is shown for comparison in Table 2.

Storage in moist air: After just 90 minutes, the proportion of apoptotic tissue cells was found to be 31%, a ten-fold increase. The percentage of apoptotic cells after 24 hours was 24%; after 48 hours, the percentage of dead cells was 39%.

Storage in PBS: After a storage time of 90 minutes, the samples show an increase in the dead cells to 17%. After 18 hours, the proportion of apoptotic cells was 8%. After 24 and 48 hours, the percentage of apoptotic cells was 10% and 18% respectively.

Storage in nutrient solution (MM0): After 90 minutes the tissue hardly shows any differences in comparison with immediate fixation. The proportion of apoptotic cells increases slightly from 3.1% to 5.1%. MM0 had fewer damaged cells than the other storage media after 90 minutes and after 18 hours. After 18 hours the proportion of dead cells was 4.3%, and after 24 hours it had increased to 14%. After 48 hours, a value of 6% was measured.

Storage in PBS+0.1%-HA and D-fructose: After a storage period of 90 minutes, the percentage of apoptotic cells was 14%; after 18 hours, 4.3%; after 24 hours, 10% and after 48 hours, 9%.

Storage in PBS+0.25%-HA and D-fructose: After a storage period of 90 minutes, the percentage of apoptotic cells was 8%; after 18 hours, 7%; after 24 hours, 13% and after 48 hours, 3%.

Table 2 shows the mean apoptosis rates for cartilage tissue in different media in tabular form. Standard deviations are given in brackets.

TABLE 2
Cell death rates for cartilage tissue stored in different media over time

| | | Storage Media | | | | |
|---|---|---|---|---|---|---|
| | No Storage | | | | PBS + 0.1% | PBS + 0.25% |
| TIME | medium | Moist Air | PBS | MM0 | HA | HA |
| Immediate | 3.1 (0.8) | — | — | — | — | — |
| 1.5 h | — | 31.1 (4.9) | 17.3 (2.3) | 5.1 (1.7) | 13.7 (2.8) | 7.8 (4.1) |
| 18 h | — | 30.5 (6.3) | 8.0 (2.6) | 4.3 (1.1) | 4.3 (2.1) | 7.2 (10.1) |
| 24 h | — | 23.7 (7.9) | 10.1 (5.2) | 13.9 (3.7) | 10.1 (3.6) | 12.6 (4.6) |
| 48 h | — | 38.8 (12.4) | 18.0 (5.1) | 6.3 (1.2) | 9.3 (3.4) | 3.3 (1.4) |

This table shows the mean cell death (apoptosis/necrosis) rates for cartilage tissue stored in different media over time in % (sd). For example cartilage tissue stored in PBS+0.25% HA solution after 48 hours was found to have 3.3% (sd 1.4) dead cells.

Example of Handling of Tendon Tissue ex vivo

Immediate fixation: The proportion of dead tendon cells when the tissue undergoes immediate fixation is 0.6% and is shown for comparison in Table 3 below.

Storage in moist air: After 90 minutes, the tissue shows a proportion of 2.0% apoptotic cells. This increases to 7.7% after 18 hours. The proportion of apoptotic cells at 24 and 48 hours is 6.6% and 10% respectively

Storage in PBS: After 90 minutes the proportion of dead cells is 0.9%. At 18 hours, this proportion has increased to 2.6%; after 24 hours, 2.7% and after 48 hours, 5.1%.

Storage in nutrient solution (MM0): After 90 minutes the proportion of dead cells is 2.1%. At 18 hours, this proportion has increased to 2.7%; after 24 hours, 3.3% and after 48 hours, 16.7%.

Storage in Nutrient Solution PBS+0.1%-HA and D-fructose: After 90 minutes the proportion of dead cells is 0.6%. At 18 hours, this proportion has increased to 2.0%; after 24 hours, 2.7% and after 48 hours, 3.8%.

Storage in Nutrient Solution PBS+0.25%-HA and D-fructose: After 90 minutes the proportion of dead cells is 0.7%. At 18 hours, this proportion has increased to 4%; after 24 hours, 4.1% and after 48 hours, 1.3%.

Table 3 shows the mean apoptosis rates for tendon tissue in different media in tabular form. Standard deviations are given in brackets.

TABLE 3
Cell death rates for tendon tissue stored in different media over time

| | | Storage Media | | | | |
|---|---|---|---|---|---|---|
| | No Storage | | | | PBS + 0.1% | PBS + 0.25% |
| TIME | medium | Moist Air | PBS | MM0 | HA | HA |
| Immediate | 0.6 (0.5) | — | — | — | — | — |
| 1.5 h | — | 2.0 (1.4) | 2.0 (1.4) | 2.1 (0.8) | 0.6 (0.8) | 0.7 (1.5) |
| 18 h | — | 7.7 (2.4) | 7.7 (2.4) | 2.7 (2.2) | 2.0 (1.8) | 4.0 (1.6) |
| 24 h | — | 6.6 (6.1) | 6.6 (6.1) | 3.3 (2.0) | 2.7 (1.3) | 4.1 (2.2) |
| 48 h | — | 10.4 (5.3) | 10.4 (5.3) | 16.7 (5.6) | 3.8 (1.7) | 1.3 (0.9) |

This table shows the mean cell death (apoptosis/necrosis) rates for tendon tissue stored in different media over time in % (sd). For example tendon tissue stored in PBS+0.25% HA solution after 48 hours was found to have 1.3% (sd 0.9) dead cells.

Additional media examples for handling MSK tissues ex vivo

1. PBS+HA−0.5%+D-fructose 3,600 mg/L: In one embodiment the hyaluronan is present in the medium at a concentration of 0.5% by weight and D-fructose is present in a concentration of 3,600 mg/L.

2. PBS+HA−1%+D-fructose 3,600 mg/L: In one embodiment the hyaluronan is present in the medium at a concentration of 1% (wt/vol) and D-fructose is present in a concentration of 3600 mg/L.

3. PBS+HA−2%+D-fructose 3,600 mg/L: In one embodiment the hyaluronan is present in the medium at a concentration of 2% (wt/vol) and D-fructose is present in a concentration of 3,600 mg/L.

4. PBS+HA−0.25%+beta-glucan−0.25%+D-fructose 3,600 mg/L: In one embodiment the hyaluronan is present in the medium at a concentration of 0.25% (wt/vol), beta-glucan is present at a concentration of 0.25% (wt/vol), and d-fructose is present in a concentration of 3,600 mg/L.

5. PBS+HA−0.25%: In one embodiment the hyaluronan is present in the medium at a concentration of 0.25% (wt/vol) without an additional saccharide.

6. PBS+HA−0.5%: In one embodiment the hyaluronan is present in the medium at a concentration of 0.5% by weight without an additional saccharide.

7. PBS+HA−0.25%+beta-glucan−0.25%: In one embodiment the hyaluronan is present in the medium at a concentration of 0.25% by weight, beta-Glucan is present at a concentration of 0.25% by weight.

8. PBS+HA−0.5%+beta-glucan−0.5%: In one embodiment, the hyaluronan is present in the medium at a concentration of 0.5% by weight, beta-glucan is present at a concentration of 0.5% by weight.

9. PBS+HA−0.25%+chitosan−0.25%: In one embodiment the hyaluronan is present in the medium at a concentration of 0.25% by weight, chitosan is present at a concentration of 0.25% by weight.

10. PBS+HA−0.25%+chitosan−0.25%+D-fructose 3,600 mg/L: In one embodiment the hyaluronan is present in the medium at a concentration of 0.25% by weight, chitosan is present at a concentration of 0.25% by weight and d-fructose is present in a concentration of 3,600 mg/L.

11. PBS+HA−0.25%+beta-glucan−0.25%+chitosan−0.25%: In one embodiment the hyaluronan is present in the medium at a concentration of 0.25% by weight, beta-glucan is present at a concentration of 0.25% by weight, and chitosan is present at a concentration of 0.25% by weight.

12. PBS+HA−0.25%+carboxymethyl-chitosan−0.02%+D-fructose 3,600 mg/L: In one embodiment the hyaluronan is present in the medium at a concentration of 0.25% by weight, carboxymethyl-chitosan is present at a concentration of 0.02% by weight and d-fructose is present in a concentration of 3,600 mg/L.

13. PBS+HA−0.25%+carboxymethyl-chitosan−0.1%+D-fructose 3,600 mg/L: In one embodiment the hyaluronan is present in the medium at a concentration of 0.25% by weight, carboxymethyl-chitosan is present at a concentration of 0.1% by weight and d-fructose is present in a concentration of 3,600 mg/L.

14. PBS+HA−0.1%+carboxymethyl-chitosan−0.1%+D-fructose 3,600 mg/L: In one embodiment the hyaluronan is present in the medium at a concentration of 0.1% by weight, carboxymethyl-chitosan is present at a concentration of 0.1% by weight and d-fructose is present in a concentration of 3,600 mg/L.

15. PBS+HA−0.1%+carboxymethyl-chitosan−0.02%+D-fructose 3,600 mg/L: In one embodiment the hyaluronan is present in the medium at a concentration of 0.1% by weight, carboxymethyl-chitosan is present at a concentration of 0.02% by weight and d-fructose is present in a concentration of 3,600 mg/L.

The object of this disclosure is to provide a tissue/cell protective media, which can improve the storage conditions for musculoskeletal tissues in comparison to the methods currently used in practice. The influence of the media on the percentage of surviving tissue cells after a given period of time was the key criterion in the assessment. The results indicated that for all investigated tissues, the solution containing hyaluronan in addition to PBS buffer and D-fructose was most suitable for maintaining the cells in a viable state. Compared to the other media investigated, this superiority was evidenced primarily by the fact that, up to a period of at least 48 hours, the referenced solution was able to minimize external stress factors in such a way that the tissue structures for all three tissues were substantially maintained up to the end, and the cells for the most part retained vitality.

A key criterion for the evaluation was the vitality of the cells after being stored in a medium for up to 48 hours. The viability of the cells on the one hand was morphologically evaluated by HE staining, and on the other hand was quantitatively determined using an apoptosis/necrosis stain.

In addition to storage in saline solutions, storage under moist, sterile surgical towels represents a generally practiced clinical standard. The results obtained using this storage method were used herein as comparative values for investigating other media.

The obtained results demonstrate that the hyaluronan-containing aqueous solutions used in these experiments had a significant influence on the vitality of the cells for example for bone, tendon and cartilage tissues. These solutions were superior over PBS, MM0 and moist surgical towel storage.

Surprisingly, it was found that MSK tissues stored in aqueous HA solutions had the lowest necrosis/apoptosis rates over the entire time. For bone stored in 0.1% HA solution, the necrosis rate at 48 hrs was 22%; cartilage and tendons stored in 0.25% HA solution had necrosis rates at 48 hrs of 3% and 4% respectively. Intermediate results were obtained using PBS without fructose and HA, followed by MM0 solution. Moist towel necrosis rates for bone, cartilage, and tendon were 65%, 39%, and 10% respectively. Storage in moist air produced the highest necrosis rates after 90 minutes and during the entire investigation time up to 48 hours. In conclusion, it was found that for example musculoskeletal tissues such as bone, tendon and cartilage should be stored in aqueous HA solutions ex vivo to maximize cell viability and minimize necrosis and apoptosis.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

What is claimed is:

1. A medium for handling tissues or cells of the musculoskeletal system, comprising an aqueous solution of hyaluronan and a first saccharide, polyol, or combination thereof, wherein the first saccharide is not hyaluronan, wherein hyaluronan is present in the medium at a concentration of about 500 mg/L to about 20,000 mg/L, and wherein the medium comprises the first saccharide, polyol, or combination thereof at a concentration of about 500 to about 10,000 mg/L.

2. The medium of claim 1, wherein the first saccharide comprises fructose, alpha-glucan, beta-glucan, chitin, chitosan, or a combination thereof.

3. The medium of claim 1, wherein hyaluronan is present in the medium at a concentration of about 1,000 mg/L to about 10,000 mg/L, and wherein the medium comprises the first saccharide, polyol, or combination thereof is D-fructose at a concentration of about 2,000 to about 4,000 mg/L.

4. The medium of claim 1, wherein the first saccharide comprises fructose and the composition comprises a second saccharide or polyol.

5. The medium of claim 1 wherein the hyaluronan has a mean molecular weight of about 100,000 Da to about 5,000,000 Da.

6. The medium of claim 1, wherein the medium further comprises one or more compounds selected from the group consisting of buffer substances, salts, vitamins, amino acids, lipids, phospholipids, antibiotics, antifungal drugs, corticosteroids, growth factors, detergents, pH indicators, and combinations thereof.

7. The medium of claim 1, wherein the pH of the medium is about 6 to about 9.

8. A method for handling musculoskeletal tissue or cells, comprising contacting musculoskeletal tissue or cells ex vivo with a medium comprising an aqueous solution of hyaluronan and a first saccharide, polyol, or combination thereof, wherein the first saccharide is not hyaluronan.

9. The method of claim 8, wherein the handling includes storing, maintaining, cultivating, coating, impregnating, preserving, or a combination thereof.

10. The method of claim 8, wherein the musculoskeletal tissue or cells are from bone, cartilage, tendon, ligament, muscle, connective, nerve, blood vessel, skin, fascia, bursa sac, joint capsule, or a combination thereof.

11. The method of claim 8, wherein the method further comprises obtaining the musculoskeletal tissue or cells.

12. The method of claim 11, wherein the tissue or cells are obtained from a human source.

13. The method of any one of claim 11, wherein the tissue or cells are obtained by harvesting during surgery.

14. The method of claim 13, wherein the tissues or cells are harvested from a dead individual.

15. The method of claim 8, wherein the contacting comprises submerging the musculoskeletal tissue or cells in the medium.

16. The method of claim 8, wherein contacting comprises pipette dripping or spraying the medium onto the musculoskeletal tissue or cells.

17. The method of claim 8, wherein the method further comprises transplanting the musculoskeletal tissue or cells into a patient.

18. A method for maintaining the viability of musculoskeletal tissue or cells ex vivo comprising contacting the musculoskeletal tissue or cells ex vivo with a medium comprising an aqueous solution of hyaluronan and a first saccharide, polyol, or combination thereof, wherein the first saccharide is not hyaluronan.

19. The method of claim 18, wherein the method further comprises maintaining said tissue or cells in contact with said medium for about 20 minutes to about 72 hours.

20. An ex vivo preserved biological tissue comprising viable musculoskeletal tissue within a medium according to claim 1.

21. The preserved tissue of claim 20, wherein the musculoskeletal tissue is from bone, cartilage, tendon ligament, muscle, nerve, blood vessel, skin, fascia, bursa, joint capsule, or a combination thereof.

22. The preserved biological tissue of claim 21, wherein the tissue is in at least one of a hypothermic condition and in an un-physiological site.
